(19) United States
(12) Patent Application Publication
(10) Pub. No.: US 2022/0193349 A1
(43) Pub. Date: Jun. 23, 2022

(54) DEVICE FOR DISPENSING A FLUID PRODUCT

(71) Applicant: APTRA FRANCE SAS, Le Neubourg (FR)

(72) Inventor: Baptiste LEBEL, Val De Reuil (FR)

(73) Assignee: APTRA FRANCE SAS, Le Neubourg (FR)

(21) Appl. No.: 17/432,670

(22) PCT Filed: Feb. 18, 2020
(86) PCT No.: PCT/FR2020/050292

§ 371 Date: Aug. 20, 2021

(30) Foreign Application Priority Data
Feb. 22, 2019 (FR) 1901825

(51) Int. Cl. A61M 11/00; B05B 11/02; B05B 11/00; A61M 15/08; A61M 15/00

(52) U.S. Cl. CPC A61M 11/007; B05B 11/025; A61M 15/004; A61M 15/08; B05B 11/3056

(57) ABSTRACT

Device for dispensing a fluid product comprising a body (1) having a dispensing opening (12) and a piercing element (4), said body (1) containing a reservoir (10) containing at least two doses of fluid product, said reservoir (10) having a proximal axial opening closed by a membrane (30) and a distal axial opening closed by a piston (20), mounted in a sliding manner in said reservoir (10) in order to dispense a dose of fluid product on each actuation, said reservoir (10) being axially movable with respect to said body (1) between a rest position, in which said piercing element (4) does not pass through said membrane (30), and a dispensing position, in which said piercing element (4) passes through said membrane (30), said membrane (30) being capable of reclosing in a sealed manner after each actuation, when said piercing element (4) comes back out from said reservoir (10), said device comprising a lateral actuation system having an actuating member (50), laterally movable with respect to said body (1) between a rest position and an actuating position, said actuating member (50) being resiliently biased towards the rest position thereof, said actuating member (50) comprising a first cam (51) cooperating with said reservoir (10) and a second cam (52) cooperating with said piston (20), such that the movement of said actuating member (50) to the actuating position thereof first moves said reservoir (10) to the dispensing position thereof then causes said piston (20) to slide in said reservoir (10), to dispense a dose of fluid product.

The present invention relates to a device for dispensing a fluid product, in particular in the form of a nasal spray.

Device for dispensing a fluid product are well known in the state of the art.

Generally, such devices comprise a reservoir containing several doses of fluid to be dispensed, and on which there is mounted a dispenser member, such as a metering pump, for dispensing a dose on each actuation. A drawback of metering pumps relates to priming, which requires one or more prior actuations before guaranteeing that a complete dose is dispensed. This problem exists not only before the first actuation, but it may also exist before each actuation, especially when, between two successive actuations, there is a lapse of time, typically several days. Unfortunately, with certain fluids, such as dangerous or particularly expensive pharmaceuticals, typically for anti-migraine treatments, such priming actuations are not desirable, since they necessarily dispense a small amount of fluid, leading to risks of poor metering for the user and/or of wasting an active agent that is expensive. For example, for an anti-migraine treatment requiring four doses of medication to be dispensed in succession, it is necessary to fill the reservoir with practically five or six doses of fluid in order to guarantee that four doses are dispensed completely. The increased cost of active agent is thus very high.

Devices also exist comprising a reservoir containing fluid to be dispensed, and a piston that is mounted in a sliding manner in said reservoir, and that is moved for selectively dispensing the fluid contained in said reservoir. When the reservoir contains a plurality of doses of fluid to be dispensed during a plurality of successive actuations, the piston is moved in a plurality of successive actuation strokes, such that a first dose is dispensed during a first actuation, a second dose is dispensed during a second actuation, etc. With that type of multi-dose device, there exists a problem of the fluid that remains in the reservoir after the first use being spoilt. Specifically, during the first actuation, the reservoir is opened, such that its content can become contaminated, in particular when the device is stored between two successive actuations. Unfortunately, depending on the type of fluid product that is dispensed by the device, in particular when it is a medication, it may be important to avoid any risk of contamination.

To overcome this drawback, document WO2019008264 proposes isolating the fluid between two actuations. However, this device may have drawbacks. Thus, the actuation may require several separate actions of the user, which may prove difficult in certain situations. In addition, the axial size may be relatively large. The metering accuracy may be dependent on the force exerted by the user during actuation.

Documents FR2847834, WO2015024653, US2005029288, US2007000950, and US2008210229 describe other devices of the state of the art.

The present invention aims to create a device for dispensing a fluid product which does not reproduce the aforementioned drawbacks.

The present invention also aims to provide a device for dispensing a fluid product of which the actuation is secure and reliable on each actuation.

The present invention also aims to provide a device for dispensing a fluid product of which the metering accuracy is improved on each actuation.

The present invention also aims to provide a device for dispensing a fluid product which avoids any fluid product being wasted before and/or after each actuation.

The present invention also aims to provide a device for dispensing a fluid product that ensures the fluid product is protected between two actuations.

The present invention also aims to provide such a device for dispensing a fluid product that is simple and inexpensive to manufacture and to assemble.

The present invention therefore aims for a device for dispensing a fluid product comprising a body provided with a dispensing opening and with a piercing element, said body containing a reservoir containing at least two doses of fluid product, said reservoir comprising a proximal axial opening closed by a membrane and a distal axial opening closed by a piston, mounted in a sliding manner in said reservoir so as to dispense a dose of fluid product on each actuation, said reservoir being axially movable with respect to said body between a rest position, in which said piercing element does not pass through said membrane, and a dispensing position, in which said piercing element passes through said membrane, said membrane being capable of reclosing in a sealed manner after each actuation, when said piercing element comes back out from said reservoir, said device comprising a lateral actuation system having an actuating member, laterally movable with respect to said body between a rest position and an actuating position, said actuating member being resiliently biased towards its rest position, said actuating member comprising a first cam cooperating with said reservoir and a second cam cooperating with said piston, such that that the movement of said actuating member towards its actuating position first moves said reservoir towards its dispensing position, then causes said piston to slide in said reservoir, to dispense a dose of fluid product.

Advantageously, said actuating member is pivotally mounted on a lower body portion, fixed to said body.

Advantageously, said actuating member is resiliently biased towards its rest position by a return spring.

Advantageously, said first cam and said second cam are formed by grooves or slots made in said actuating member.

Advantageously, said first cam cooperates with at least one lug integral with said reservoir, said lug sliding in said first cam during the actuation, and said second cam cooperates with at least one lug integral with said piston, said lug sliding in said second cam during the actuation.

Advantageously, said first cam comprises a first axially oblique portion and a second substantially horizontal portion, said first portion cooperating with said lug at the beginning of the actuation in order to push it axially upwards, which moves said reservoir axially upwards, causing said piercing element to pierce said membrane, and said second portion cooperates with said lug at the end of the actuation, such that, after piercing said membrane, said reservoir is not pushed upwards either.

Advantageously, said lug is formed on a support member fixed to said reservoir.

Advantageously, said second cam comprises a first portion and an axially oblique second portion, said first portion cooperating with said lug at the beginning of the actuation such that said lug does not undergo any axial force by said cam and does not move at the beginning of said actuation, and said second portion cooperating with said lug at the end of the actuation so as to push said lug axially upwards, which moves said piston axially upwards in said reservoir so as to dispense a dose of the fluid product contained in said reservoir.

Advantageously, said first portion is wider than said lug.

Advantageously, said lug is formed on an actuator sleeve cooperating with a piston rod fixed to said piston.

Advantageously, said piston rod comprises a set of teeth that cooperate with at least one hook of said actuator sleeve.

Advantageously, an indication element is fixed to said piston rod, said indication element being visible in a viewing window, advantageously provided with indication indices such as graduations and/or alphanumeric and/or coloured indices.

Advantageously, said actuating member comprises a deformable projection cooperating at the beginning of the actuation with an abutment of the body in order to accumulate energy in the user's hand.

Advantageously, said piercing element is integral with an insert fixed in said body, upstream from said dispensing opening.

Advantageously, a spray profile is provided directly upstream from said dispensing opening.

These advantages and characteristics of the present invention, and others, will appear more clearly from the following detailed description, given by way of non-limiting examples, and with reference to the accompanying drawings, and in which:

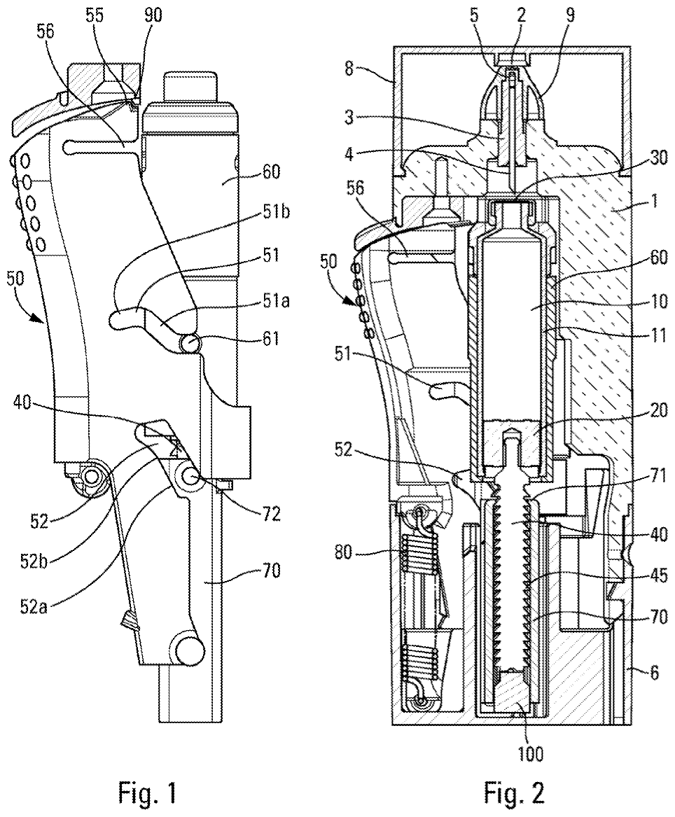

FIG. 1 is a side, partial schematic view of a device for dispensing a fluid product according to an advantageous embodiment of the present invention, in the rest position,

FIG. 2 is a cross-sectional view of the device of FIG. 1,

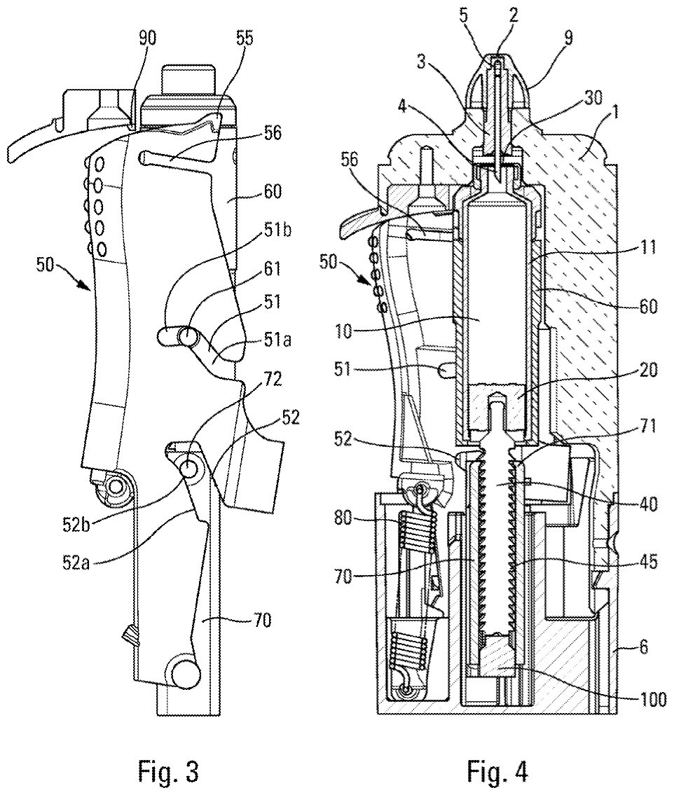

FIG. 3 is a view similar to that of FIG. 1, during actuation, after the reservoir has been pierced, and before the first dose has been dispensed,

FIG. 4 is a cross-sectional view of the device of FIG. 3,

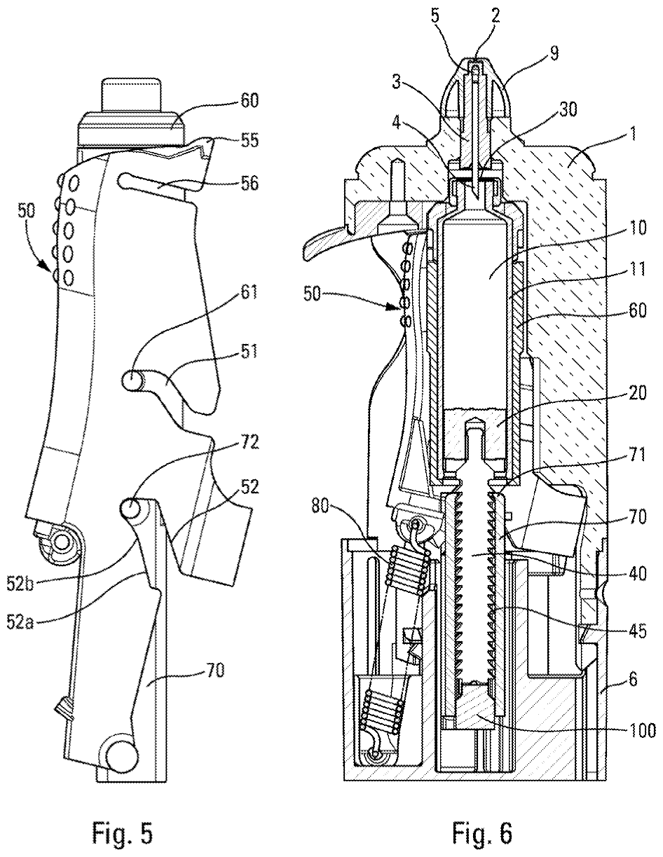

FIG. 5 is a view similar to that of FIG. 3, during actuation, during the dispensing of a dose,

FIG. 6 is a cross-sectional view of the device of FIG. 5,

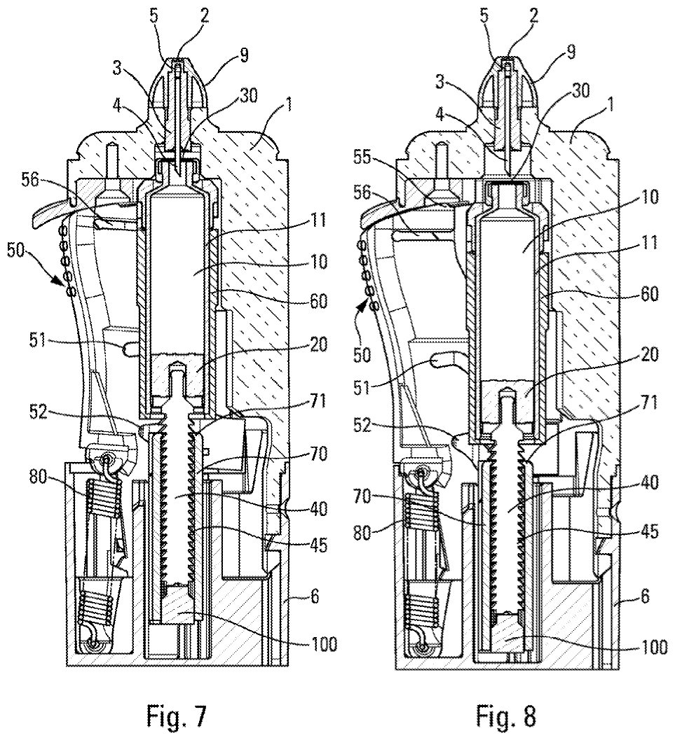

FIG. 7 is a view similar to that of FIG. 6, during actuation, after the dispensing of a dose and before the closing of the reservoir,

FIG. 8 is a view similar to that of FIG. 7, after actuation, with the device ready for the next actuation, and

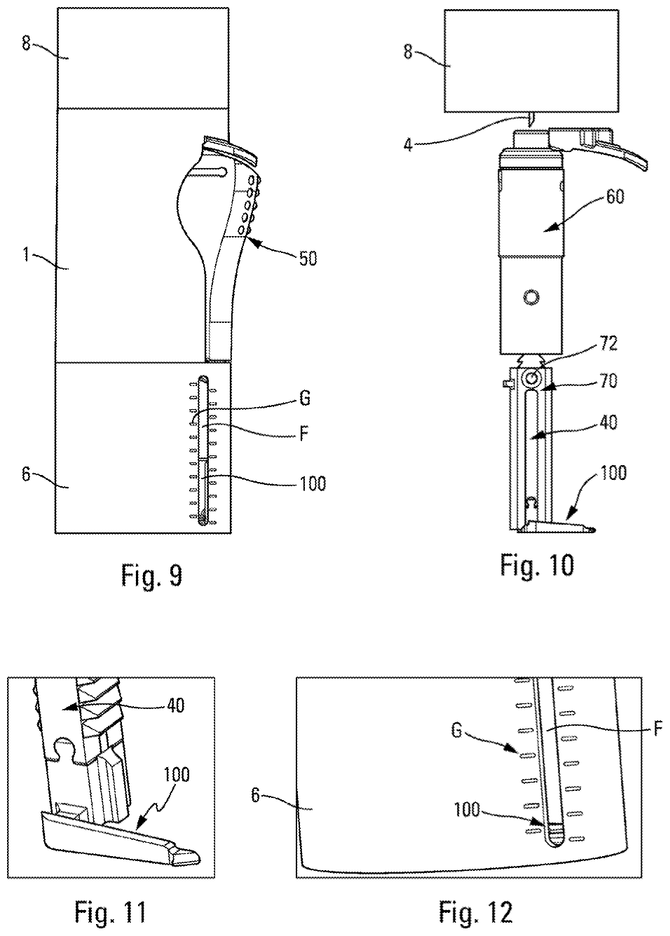

FIGS. 9 to 12 are schematic views illustrating the dose indicator according to an advantageous embodiment.

The present invention will be described below with reference to the schematic embodiment. The shapes and dimensions of the various component parts are only illustrative and not limiting. It is understood that the present invention applies more generally to any type of device containing at least two doses

The terms “proximal” and “distal” refer to the dispensing opening. The term “axial” refers to the longitudinal central axis of the reservoir. The terms “top”, “bottom” and “horizontal” refer to the upright position of the device shown in the Figures.

With reference to the figures, the dispenser device comprises a body 1 that is provided with a dispensing opening 2. Optionally, as in the example shown, the dispensing opening 2 is made in a dispenser head 9 fixed on the body 1. A removable cover 8 may be provided for protecting said dispensing opening 2 when the device is not used.

Said body 1 contains a reservoir 10 that contains a plurality of doses of fluid product, in particular a liquid medication, for example for spraying into a user's nose. This reservoir 10 advantageously comprises a hollow body 11 that is mainly cylindrical, comprising a proximal axial opening and a distal axial opening. The distal opening is closed in a sealed manner by a piston 20, mounted in a sliding manner in said reservoir 10. The proximal opening is closed by a membrane 30, preferably sealed. The drawings show a proximal opening formed on a cylindrical portion of smaller diameter, but this is not essential, and the hollow body 11 forming the reservoir 10 could also be completely cylindrical.

Said piston 20 cooperates with a piston rod 40 that extends axially out from said reservoir 10. Said piston rod 40 therefore cooperates, on the one hand, with said piston 20, and it cooperates, on the other hand, with a lateral actuation system, as will be described in more detail below.

The piston rod 40 is notched, i.e. it comprises toothing 45 extending longitudinally along said piston rod. An actuator sleeve 70 is mounted around said piston rod 40, said actuator sleeve 70 comprising at least one hook 71 cooperating with said toothing 45 of said piston rod. Preferably, as can be seen in the figures, this hook 71 is formed at the proximal axial end of said actuator sleeve 70. The actuator sleeve 70 further comprises at least one lug 72 of which the function will be described in more detail below.

An advantageous dose indicator is shown in FIGS. 9 to 12. This indicator advantageously comprises an indication element 100 fixed to the piston rod 40, preferably at its distal axial end, and therefore moving axially together with it. A viewing window F made in a lower body portion 6 fixed to the body 1 makes it possible to view the movement of said indication element 100 on each actuation. Preferably, said window F is provided with indication indices, such as, for example, graduations G. In a variant, it could also comprise alphanumeric and/or coloured indices. Other variants may also be considered.

The body 1 and/or the dispensing head 9 may comprise, upstream from said dispensing opening 2, an insert 3 supporting a hollow piercing element 4, such as a needle or a cannula, intended to pierce said membrane 30 of the reservoir 10.

Said insert 3 may also define a spray profile 5 directly upstream from said dispensing opening 2, so as to generate a spray, in particular a nasal spray. This spray profile 5 may be formed conventionally in the proximal axial edge of said insert 3 and/or in the bottom wall of said body 1, and/or of said dispensing head 9, surrounding said dispensing opening 2 and facing said proximal axial edge of the insert 3. This spray profile 5 may comprise swirl channels and a swirl chamber.

The reservoir 10 is mounted to slide axially in said body 1 between a rest position, in which said piercing element 4 does not pass through said membrane 30, and a dispensing position, in which said piercing element 4 passes through said membrane 30. The reservoir 10 may be fixed in a support member 60, which may in particular surround said reservoir 10 as shown in the example in the figures. The support member 60 further comprises at least one lug 61 having a function that will be described in greater detail below. In a variant, said at least one lug 61 could be formed directly on the reservoir 10.

In order to perform successive actuations of the device, a lateral actuation system is provided.

This lateral actuation system comprises an actuating member 50, laterally movable with respect to said body 1 between a rest position, shown in FIGS. 1 and 2, and an actuating position, shown in FIGS. 5 and 6. In the example in the figures, this actuating member 50 is pivotally mounted on a lower body portion 6, fixed to said body 1.

This actuating member 50 comprises a first cam 51 and a second cam 52, preferably formed by grooves or slots.

The first cam 51 cooperate with said lug 61 which slides in the groove 51 during the actuation. The second cam 52 cooperates with said lug 72 which slides in the groove 52 during the actuation.

Advantageously, the groove 51 comprises a first portion 51a and a second portion 51b. The first portion 51a cooperates with said lug 61 at the beginning of the actuation, and the second portion 51b cooperates with said lug 61 at the end of the actuation.

Preferably, the first portion 51a is axially oblique, such that when the user pivots the actuating member 50 from its rest position towards its actuation position, the lug 61 is axially pushed upwards by sliding in said first portion 51a, which axially moves said reservoir 10 upwards, causing the piercing of the membrane 30 by the piercing element 4, as can be seen in FIGS. 1 to 4. Advantageously, the second portion 51b is substantially horizontal, such that after piercing the membrane 30, the reservoir 10 is not pushed upwards any more.

Preferably, the second groove 52 comprises a first portion 52a and a second portion 52b. The first portion 52a cooperates with said lug 72 at the beginning of the actuation, and the second portion 52b cooperates with said lug 72 at the end of the actuation.

Advantageously, when it moves in the first portion 52a, said lug 72 does not undergo any axial force by said cam 52, such that when the user pivots the actuating member 50 from its rest position towards its actuating position, the lug 72 does not move at the beginning of said actuation, as can be seen in FIGS. 1 and 2. Advantageously, said first portion 52a is wider than said lug 72. When the lug 72 comes into contact with the lower wall of the second cam 52, it then cooperates with the second portion 52b of said cam, as can be seen in FIGS. 3 to 6. Advantageously, the second portion 52b is axially oblique, such that when the user causes the actuating member 50 to pivot further towards its actuation position, the lug 72 is pushed axially upwards by sliding in said second portion 52b, which moves said actuator sleeve 70 axially upwards, causing the piston rod 40 to move axially upwards, under the effect of the push exerted by the hook(s) 71 on the toothing 45. This axial movement of the piston rod 40 moves the piston 20 in the reservoir 10 and therefore generates the dispensing of a dose of the fluid product contained in the reservoir.

A return spring 80 is mounted between the actuating member 50 and the body 1, so as to return said actuating member 50 into its rest position after each actuation.

The operation of the device shown in the figures is as follows.

In the rest position of FIGS. 1 and 2, the reservoir 10 is isolated from the atmosphere, on the one hand, by the piston 20 and on the other hand, by the membrane 30.

When the user presses on the actuating member 50, it first moves the reservoir 10 with respect to the body 1, to this pierce the membrane 30 by means of the piercing element 4.

Continuing the movement of the actuating member 50 towards the dispensing opening 2 will then cause the piston 20 to move inside the reservoir 10, and therefore causes a dose of fluid product to be dispensed. The fluid product is therefore pushed by said piston 20 through the piercing element 4 towards the spray profile 5, then out from the device through the dispensing opening 2.

Thus, it is the sizing of the toothing 45 that defines the dose of fluid product, and it suffices to modify this toothing on the piston rod 40 in order to modify the metering, without having to modify the rest of the device. Likewise, the number of doses that can be dispensed can be easily modified, corresponding to the number of teeth in the toothing 45.

Advantageously, at the beginning of the actuation, the actuating member 50 cooperates with the body 1 in order to accumulate energy in the user's hand. Only by exceeding a predetermined force threshold can the actuating member 50 be moved towards its actuating position, the energy thus accumulated in the user's fingers making it possible to generate sufficient force to first pierce the membrane 30 and then to move the piston 20 in the reservoir 10, thus guaranteeing that the complete dose is dispensed. Advantageously, this accumulation of energy can be achieved by a deformable projection 55 of the actuating member 50 which cooperates with an abutment 90 of the body 1. This abutment 90 may be formed directly on the body 1 or on a portion fixed to said body 1. The deformable projection 5 may be defined by a cutout 56 which allows said deformable projection to be deformed. In the example shown, said cutout 56 is substantially horizontal, such that the deformation of the projection 55 is substantially axial. Other implementations are, however, also possible for achieving this energy accumulation at the beginning of the actuation.

It must be noted that the cams 51 and 52 can be modified to customise the actuation sequence.

After a dose of fluid product has been dispensed, the device is in the position shown in FIG. 6.

When the user releases the pressure on the actuating member 50, the spring 80 will return it to its rest position.

At the beginning of the return stroke, the lug 61 moves into the second portion 51b of the first cam 51, without this generating an axial movement for the reservoir 10. However, the lug 72 will be pushed downwards by the second portion 52b of the second cam 52, which will move the actuator sleeve 70 axially downwards, with the hook 71 which will snap into the next tooth of the toothing 45, as can be seen in FIG. 7.

Advantageously, the toothing 45 of the piston rod 40 cooperates with an appropriate non-return ratchet, which allows the axial upward movement of the piston rod 45 and blocks its axial downward movement. Thus, when the actuating member 50 moves from its rest position towards its actuating position, the hook 71 pushes on a tooth of the piston rod 40 so as to move it axially upwards, with the non-return ratchet passing behind the next tooth. Similarly, when the actuating member 50 returns from its actuating position to its rest position, the non-return ratchet blocks the downward axial movement of the piston rod 40, and the hook 71 passes behind the next tooth of the toothing 45.

At the end of the return stroke, the lug 72 moves in the first portion 52b of the second cam 52, which no longer generates axial movement for the actuator sleeve 70. However, the lug 61 will be pushed downwards by the second portion 51b of the first cam 51, which will move the reservoir 10 axially downwards, with the piercing element 4 coming out of the reservoir 1 and the membrane 30, as can be seen in FIG. 8.

When the piercing element 4 comes back out from the reservoir 10, the membrane 30 recloses, to isolate again the content of the reservoir from the atmosphere.

At the end of the return stroke, the actuating member 50 returns to its rest position, with the deformable projection 55 deforming thanks to the cutout 56 and returning to position itself against the abutment 90.

The device is thus ready for the next actuation.

Thus, thanks to the lateral actuation, the present invention makes it possible to reduce the axial dimension of the device by moving the actuating member 50 sideways. It also makes it possible to carry out the entire actuating cycle with one single action by the user, namely pressing and then releasing the actuating member 50, and therefore with one single hand.

The membrane 30 is capable of reclosing in a sealed manner after each actuation, when the piercing element 4 comes back out from the reservoir 10. This guarantees the integrity of the fluid product between two actuations.

Advantageously, the membrane 30 may be formed as a structure having two layers, with two different materials:
an inner layer made out of butyl rubber that is in contact with the fluid, that is inert relative to the fluid product, and that typically has thickness of about 0.6 mm to 0.7 mm; and
an outer layer that is made out of polyisoprene for its automatic closing property, and that typically has thickness of about 1 mm to 1.5 mm.

Advantageously, the membrane is made by calendering and cross-linking, and then by cutting out, in particular by punching. In a first variant, the two raw materials are calendered and cross-linked together. In a second variant, a first material is calendered and cross-linked, then the second material is calendered on the first material, then cross-linked.

Other materials having similar properties may be considered. Likewise, a single-layer structure, preferably made out of butyl rubber, could be considered if the number of doses to be dispensed is small, typically between 2 and 5 doses. Likewise, a membrane with more than two layers would also be possible.

The present invention has been described above with reference to an advantageous embodiment that is not limiting, and any useful modification can be applied to the present invention without moving away from its scope, as defined by the accompanying claims.

1. A device for dispensing a fluid product comprising a body having a dispensing opening and a piercing element, said body containing a reservoir containing at least two doses of fluid product, said reservoir having a proximal axial opening closed by a membrane and a distal axial opening closed by a piston, mounted in a sliding manner in said reservoir in order to dispense a dose of fluid product on each actuation, said reservoir being axially movable with respect to said body between a rest position, in which said piercing element does not pass through said membrane, and a dispensing position, in which said piercing element passes through said membrane, said membrane being capable of reclosing in a sealed manner after each actuation, when said piercing element comes back out from said reservoir, wherein said device comprises a lateral actuating system comprising an actuating member, laterally movable with respect to said body between a rest position and an actuating position, said actuating member being resiliently biased towards its rest position, said actuating member comprising a first cam cooperating with said reservoir and a second cam cooperating with said piston, such that the movement of said actuating member towards its actuating position first moves said reservoir towards its dispensing position and then causes said piston to slide in said reservoir, to dispense a dose of fluid product.

2. The device according to claim 1, wherein said actuating member is pivotably mounted on a lower body portion, fixed to said body.

3. The device according to claim 1, wherein said actuating member is resiliently biased towards its rest position by a return spring.

4. The device according to claim 1, wherein said first cam and said second cam are formed by grooves or slots made in said actuating member.

5. The device according to claim 4, wherein said first cam cooperates with at least one lug integral with said reservoir, said lug sliding in said first cam during the actuation, and said second cam cooperates with at least one lug integral with said piston, said lug sliding in said second cam during the actuation.

6. The device according to claim 5, wherein said first cam comprises an axially oblique first portion and a substantially horizontal second portion, said first portion cooperating with said lug at the beginning of the actuation to push it axially upwards, which axially moves said reservoir upwards, causing said piercing member to pierce said membrane, and said second portion cooperating with said lug at the end of the actuation, such that after piercing said membrane, said reservoir is not pushed further upwards.

7. The device according to claim 6, wherein said lug is formed on a support member fixed to said reservoir.

8. The device according to claim 5, wherein said second cam comprises a first portion and a second portion that is axially oblique, said first portion cooperating with said lug at the beginning of the actuation such that said lug does not undergo any axial force by said cam and does not move at the beginning of said actuation, and said second portion cooperating with said lug at the end of the actuation so as to push said lug axially upwards, which moves said piston axially upwards in said reservoir in order to dispense a dose of the fluid product contained in said reservoir.

9. The device according to claim 8, wherein said first portion is wider than said lug.

10. The device according to claim 8, wherein said lug is formed on an actuator sleeve cooperating with a piston rod fixed to said piston.

11. The device according to claim 10, wherein said piston rod comprises toothing (45) cooperating with at least one hook of said actuator sleeve.

12. The device according to claim 10, wherein an indication element is fixed to said piston rod, said indication element being visible in a viewing window (F), advantageously provided with indication indices such as graduations (G) and/or alphanumeric and/or coloured indices.

13. The device according to claim 1, wherein said actuating member comprises a deformable projection cooperating, at the beginning of the actuation, with an abutment of the body to generate an accumulation of energy in the user's hand.

14. The device according to claim 1, wherein said piercing element is integral with an insert fixed in said body, upstream from said dispensing opening.

15. The device according to claim 1, wherein a spray profile is provided directly upstream from said dispensing opening.
